(19) United States
(12) Patent Application Publication
(10) Pub. No.: US 2004/0215065 A1
(43) Pub. Date: Oct. 28, 2004

(54) Materials and methods for visualizing and illuminating the lens and anterior lens capsule of the eye

(76) Inventor: Gysbert Van Setten, Danderyd (SE)

Correspondence Address: Gysbert Van Setten, Parsevalstr. 20, Sankt Augustin, 53757, DE

(21) Appl. No.: 10/734,057

(22) Filed: Dec. 11, 2003

Related U.S. Application Data
(60) Provisional application No. 60/432,425, filed on Dec. 11, 2002.

(30) Foreign Application Priority Data
Dec. 12, 2002 (DE) DE 101 25 161A1

(51) Int. Cl. A61B 1/06

(52) U.S. Cl. 600/249

(57) ABSTRACT

The subject invention pertains to materials and methods for the visualization of the lens and the anterior lens capsule of the eye. In particular, the subject invention pertains to visualization of the lens and anterior lens capsule during cataract operations, with or without lens replacement procedures. The subject invention involves directing light or introducing a means for emitting light, particularly laser light, into the eye.

### CROSS-REFERENCE TO RELATED APPLICATION

This application claims the benefit of U.S. Provisional Application No. 60/432,425, filed Dec. 11, 2002, which is hereby incorporated by reference in its entirety, including all figures, tables, and drawings.

### BACKGROUND OF THE INVENTION

Opacification of the lens, also called cataract, is a very common condition affecting humans and animals. It has various causes such as age, trauma, metabolic and others. The cause may be a congenital or genetic anomaly, or be presented in association with diseases or specific medications. Independent of this cataract is associated with a decrease of visual acuity. Intense cataract may cause severe loss of visual capacity, practically blindness. Anatomically, a cataract is an opacity of the crystalline lens, the intensity, localization, and extent of which may vary. Any opacity in the usually crystal clear lens leads to impaired optical properties of the lens. As the lens is a key component of the eye's optical system, opacities may lead to light scattering, absorption and other phenomenon. The patients experience these phenomena as a decrease of visual acuity, glare, diplopia, and multiplopia. Any opaque region can prevent or retard the transmission of light through to the retina. Apart from affecting the capacity to visualize objects, cataracts may also scatter light, such that discomfort from sunlight and bright lights may result, i.e., glare. In addition to humans, cataracts also affect animals, including domesticated animals, such as dogs and cats.

Cataract surgery, which requires removal of the lens, is currently one of the most common surgical procedures performed. The standard treatment for cataracts is removal of the lens substance, and replacement of the cataractous lens with an intraocular lens. Intraocular lenses are inserted into the eye in place of the removed cataractous lens, thereby improving and frequently maintaining vision at normal levels.

Techniques for cataract treatment have been advanced by phacoemulsification surgery, which involves making a few, usually two, very small corneo-limbal incisions in order to gain access to the lens capsule, creating an incision in the lens capsule in order to access the lens substance (cortex and nucleus), and removing the lens by high frequency ultrasound and suction accompanied by irrigation. The capsule (the basement membrane which surrounds the crystalline lens) and its attachment, the zonular apparatus which suspends the lens from the ciliary body, are left largely intact, except for the anterior capsulotomy, that is the incision in the capsule through which the cataractous lens is removed and its replacement inserted. Preservation of this inert ocular structure (i.e., capsule and zonular apparatus) supplies a mechanism by which intraocular lenses can be implanted so that they do not impinge on vital structures (as occurs with angle or iris supported intraocular lenses), and thus avoids chronic complications such as uveitis (inflammation of the uvea of the eye), glaucoma, and corneal decompensation (increasing haziness of the cornea).

During phacoemulsification surgery, the anterior crystalline lens capsule is torn away to form a circular opening by which the lens material can be removed (continuous curvilinear capsulorhexis). This produces a strong capsular rim that resists tearing even when stretched—generally improving the safety margin during surgery. In extracapsular surgery, a so-called “can-opener” capsulotomy is performed, but the irregular edges of the capsule are prone to radial capsular extension tears, which can result in loss of vitreous or the lens into the vitreous, both of which are adverse events that frequently result in a suboptimal result and complications. Furthermore, following intraocular lens insertion, lens capsular fibrosis can cause decentration of the intraocular lens (with resultant astigmatism and lens-edge induced glare), but this occurs less frequently following capsulorhexis because the forces generated within the capsular bag are more symmetrical. Thus, capsulorhexis is a critical step in achieving a good surgical result.

Visualization of the capsule, particularly the anterior lens capsule, is critical in capsulorhexis procedures. Capsulorhexis is generally performed in the presence of the red reflex, where light reflecting off the retina highlights the anterior capsule against a reddened background. Capsulorhexis is difficult in patients with dense cataracts (mature, usually white or very dense cataracts) where the red reflex is difficult to see, or simply not present. In these cases, extracapsular surgery still is possible but with a higher risk of complications and difficulties. Capsulorhexis may also be difficult to perform in the presence of corneal opacities (e.g., scars) or vitreous opacity, such as vitreous hemorrhage (sometimes seen in patients with diabetes).

Various approaches have been taken to improve the success rate of performing capsulorhexis in patients with dense cataracts, or in conditions where the anterior lens capsule cannot be visualized for whatever reason.

Early attempts used side illumination light sources and high magnification, while dimming the operating theater lights, in order to enhance the view of the capsule. These procedures were cumbersome and costly. Moreover, these arrangements were not particularly effective because of the placement of the light source, the type of light source utilized, and the dependency on the surgeons expertise. Additionally, intense light shaded from outside on a highly opaque lens might lead to very irritating light reflexes and light scattering within the lens, thus hardly facilitating the procedure of capsulorrhexis.

The early use of dyes to visualize the anterior lens capsule, such as fluorescein, have also not become commonly accepted, partly due to the dyes' interaction with ocular tissues and technical difficulties of visualization. Likewise, the use of the patient's own blood (haemocolouration technique) to colour the capsule had various draw backs. In addition, development of specialized cutting devices did not completely meet the expectations for better surgical safety and flexibility.

The only technique that has recently reached a more widespread use is the utilization of dilutions and compositions with the vital dye trypan blue. However, this technique is still rather new and requires a number of fluid exchanges in the anterior chamber of the eye during surgery, which increases time spent in surgery and enhances the risk of infection. Also possible are hydrodynamic side effects on potentially sensitive structures, such as the trabecular meshwork, due to the required frequent fluid exchanges within the eye.

Some procedures involve directing light on certain ocular tissues (e.g., the retina) for the purpose of recognizing structures on the surface of the tissue at which the light is directed (see, for example, U.S. Pat. No. 5,624,438 (Turner), U.S. Pat. No. 5,630,809 (Conner), and European Patent Application No. 86301737.2 (Wok), which are incorporated herein in their entireties). Therefore, these procedures use direct (non-reflected) light to illuminate their target tissue, with as large an illumination angle as possible.

None of the aforementioned methods are able to combine time efficiency, cost efficiency, and safety in an optimal way. The need for macular protection during these procedures has not been adequately recognized. However, as the most light-sensitive tissue at the bottom of the eye, protection of the macula should be a major concern. This sensitive tissue is often altered during aging and has, particularly in the elderly, become increasingly sensitive to all kinds of manipulation and trauma. Excessive light exposure during surgery of the eye may lead to postoperative permanent loss of vision and visual acuity. During current surgical procedures, light exposure is still commonly considered unavoidable and accepted.

Hence, there remains a distinct need for methods and materials that enable ready visualization of the lens and the anterior lens capsule, so that cataract surgery and one of its most sensitive steps, capsulorhexis, can be carried out in the safest, most efficient, and cost-effective way.

### BRIEF SUMMARY OF THE INVENTION

The subject invention pertains to materials and methods for the visualization of the lens and the anterior lens capsule of the eye. In particular, the subject invention pertains to visualization of the lens and anterior lens capsule during cataract operations, with or without lens replacement procedures.

The present invention is based on the finding that a means for emitting light that is brought into the eye may be used to induce light scattering in the lens from a close proximity the reflected light of which is used to illuminate lens structures, such as the lens capsule from behind, that means by retrograde illumination. The invention allows visualize details of the lens capsule during capsuorhexis in a way that has not previously been possible. Thus, the present invention concerns a method for visualizing the lens and anterior lens capsule of the eye by introducing a light emitting means into the eye in the purpose to cause light scattering leading to illumination from more anteriorily located structures from behind. Preferably, the light emitting means is a light source that produces laser light, such as that emitted by a helium neon laser. The light emitting means emits light in a wavelength that excellently imitates a red fundus reflex and does decrease risk of breaking darkadaption of the surgeons eyes. The high light energy of the laser light does also cause strong light scattering in dens lenses making any irregularities such as folds or rifts, such as the edge of the capsulorhexis, easily visible as they will appear in this retroillumination light as a sharp, dark edge. Laser light is particularly useful in carrying out the subject invention, due to its monochromatic and coherent properties.

According to the method of the subject invention, the light emitting means (also referred to herein as the “phacoilluminator”) is directed at the eye from a direction that is, preferably, not parallel to the axis of the eye (non-coaxially). Therefore, the macula is protected from direct illumination by the light emitting means. Advantageously, because the method of the invention can be a one step procedure, compared to the other methods mentioned above, the risk for infection is decreased and a time gain during surgery is possible. Preferably, the light emitting means emits laser light of a wavelength that is known to be safe for exposure to the eye, such as that of helium neon lasers. Advantageously, this allows the method of the invention to be free of the disadvantages associated with the use of dyes or other methods mentioned above. However, that is not to say that such dyes and methods cannot be utilized with the subject invention.

In one aspect, the subject invention concerns a method for the visualization of the lens and anterior lens capsule of the eye by causing retroillumination of the lens capsule as a result of inducing light scattering after the introduction of the light emitting means into the lens of the eye. Optionally, the light emitting means can be sufficiently curved or bent, such that the emitted light is directed out of the eye, in order to provide backlighting to the lens and/or anterior lens capsule directly. In one embodiment, the method is carried out during a procedure involving removal of cataractous lenses, a lens replacement procedure, or any other surgery on the lens. In another embodiment, the method does not involve introducing a dye into the anterior chamber of the eye.

In accordance with a further aspect of this invention, there is provided a method for the reducing the exposure of the macula to the light used for the surgery of the lens.

In accordance with a further aspect of this invention, there is provided a method for the visualization of the lens and anterior lens capsule during a capsulorhexis procedure, which comprises the introduction of a light emitting means through the anterior chamber to or into the lens of the eye with or without perforating the lens capsule.

The apparatus of the subject invention comprises a light transporter (such as an optical fiber), which can be operably connected to (in operable communication with) a light source. The light transporter has a proximal end and a distal end (free end). The proximal end of the light transporter is connected to the light source, such that light energy is provided by the light source to the light transporter. Optionally, the distal end of the light transporter can include a means for handling the light transporter, such as a handpiece, which can be made of rubber, plastic, or other material. Preferably, the handling means is sufficiently ergonomic so as to minimize tension within the hand of the operator of the apparatus.

The materials and methods of the subject invention can be utilized to visualize the lens and anterior lens capsule of humans, as well as non-human animals, such as domesticated animals. For example, the subject invention can be utilized for veterinary applications on dogs, cats, and other mammals.

### BRIEF DESCRIPTION OF THE DRAWINGS

FIG. 1 shows an embodiment of the apparatus of the subject invention. In this embodiment, the means for emitting light includes a light transporter with a handpiece on its distal end, and the light transporter is connected to a light source.

FIGS. 2A-2D show a closer view of the distal end of the light transporter of the subject invention. FIG. 2A shows the distal end of the light transporter of the subject invention, with a handpiece. FIG. 2B shows another possible form which affect the shape and direction of the light beam emitted.

FIG. 3 is a schematic side view of eye, which shows the position of the lens within the eye.

FIGS. 4A and 4B show, respectively, an example of a casulorhexis procedure using conventional illumination and the phacoillumination of the subject invention.

FIG. 5 shows a light emitting means being introduced into the eye.

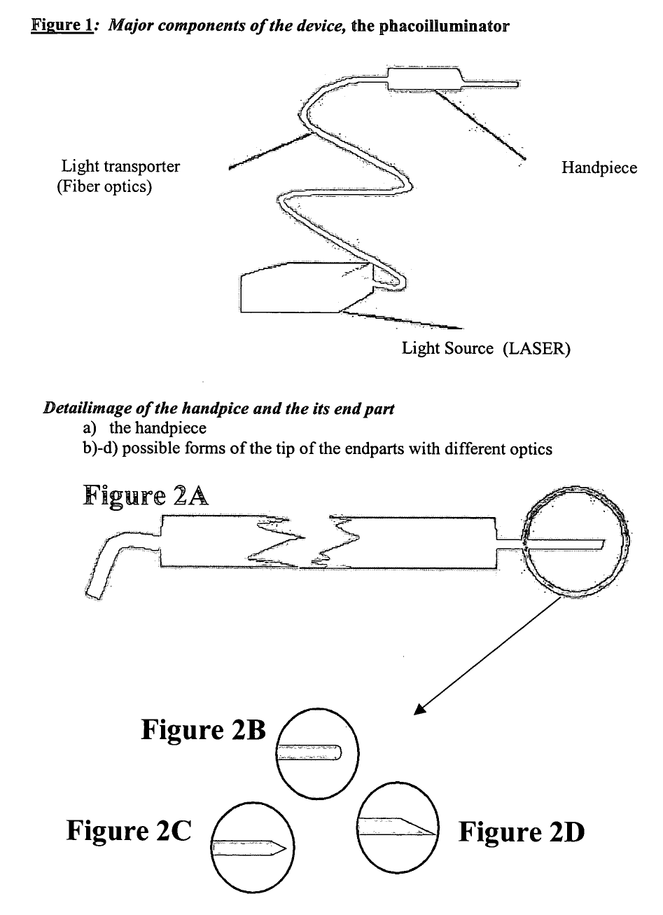

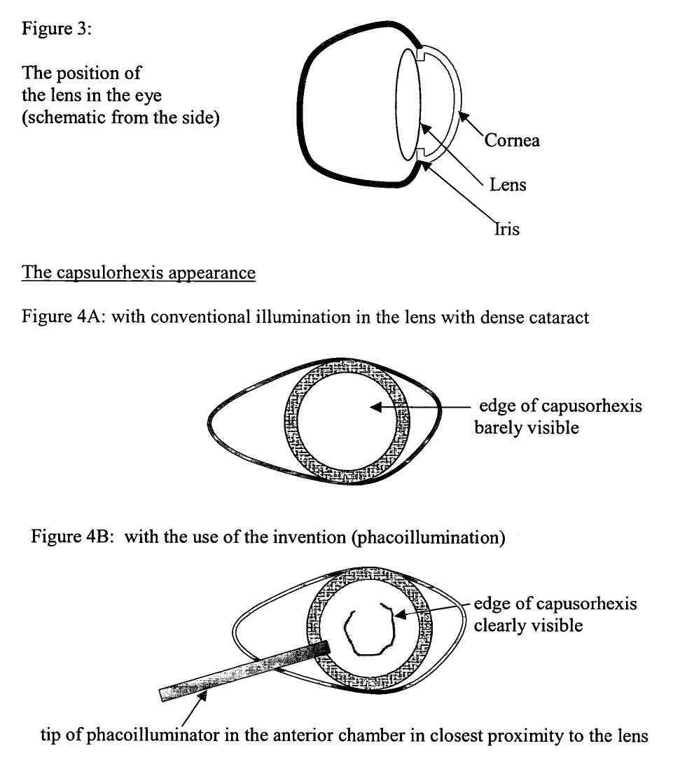

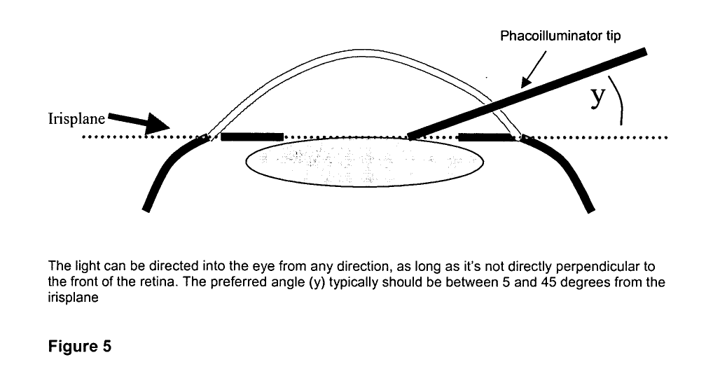

### DETAILED DISCLOSURE OF THE INVENTION

The present invention concerns a method for visualizing the lens and/or anterior lens capsule of a subject, said method comprising introducing a means for emitting light into the subject's eye, wherein light emitted by the light emitting means illuminates the lens and/or anterior lens capsule of the eye, and visualizing the illuminated lens and/or anterior lens capsule of the eye. The reflection and scattering of the emitted light brought into the lens provides backlighting to the lens and/or anterior lens capsule, i.e., retro- or retrograde illumination instead of direct or antegrade illumination). Alternatively, the light emitting means can be sufficiently curved or bent, such that the emitted light is directed out of the eye, in order to provide backlighting to the lens and/or anterior lens capsule directly.

The present invention also concerns a method for visualization of the lens and/or the anterior lens capsule of the eye during a capsulorhexis procedure, comprising introducing a viscoelastic substance into the anterior chamber of the eye, inserting a means for emitting light into the subject's eye, wherein light emitted by the light emitting means illuminates the lens and/or anterior lens capsule of the eye, and creating a capsulorhexis within the eye.

Preferably, the emitted light in the methods and apparatus of the present invention is focused, i.e., does not have a wide angle of illumination. For example, the light can have an angle of dispersion between about 0 degrees and about 20 degrees, thereby achieving a higher local scattering of the emitted light in the lens.

Thus, the methods of the present invention are readily discernable from other methods which use direct light on ocular tissues for the purpose of recognizing structures at their surfaces, using as large an illumination as possible.

The subject invention uses light, such as laser light, in methods for ocular surgery, particularly in the surgery of cataract affected lenses. Surprisingly, it has been determined that either direction of a light into the eye or introduction of a light source into the eye enable visualization of the lens and anterior lens capsule, both in clear lenses, and in lenses with cataract. In particular, laser (light amplified stimulated emission of radiation) light, such as helium neon laser light (or other light of wavelength of about 630 to about 670 nm) has been found to permit visibility of the lens surface and any irregularities of the lens, such as the edge of a capusulorhexis. Visualization is possible even in the presence of a white or black cataract and the resulting absence of a normal fundus reflex.

As the anterior capsule is readily visualized using the method of the subject invention, under the operating microscope, the lens can be subjected to capsulorhexis without the use of any use of dyes, and cataract extraction can proceed as in a routine case.

Helium neon lasers are widely used in diagnostics. For example, helium neon lasers are used as aiming beams for other lasers such as YAG (Yttrium-Aluminium Gamet), Argon lasers, or Diode lasers. However, today lasers are typically not yet used as illumination devices in medicine.

As indicated above, cataractous lenses for the surgery of which the invention shall be used may be human lenses, or lenses of animals, such as domestic animals.

According to the methods of the subject invention, the light emitting means can directed to emit light into the lens of the eye at various angles.

In another aspect, the subject invention concerns an apparatus for visualizing the lens and anterior lens of the eye. The apparatus of the subject invention comprises a light emitting means. The light emitting means can be any light generating source, such as a lamp or solid state light. Preferably, the light emitting means comprises a light transporter, such as a conductor of fiber optic light. More preferably, the light transporter is an optical fiber. The length, size, and shape of the light transporter are not critical. Materials and methods for the production of light transporters, such as optical fiber, or known to those of ordinary skill in the art.

As used herein, the term “optical fiber” is used to refer generally to any optical waveguide or structure having the ability to transmit the flow of radiant energy along a path parallel to its axis and to contain the energy within or adjacent to its surface. “Step index,” “gradient index,” and “single mode” fibers are subcategories within the optical fiber designation. The term “multimode” optical fiber refers to an optical waveguide that will allow more than one bound mode to propagate.

Step index fibers include a transparent cylindrical core of relatively high refractive index light-conducting material. Typical core materials include, silica, plastic, and glass. The core is cylindrically surrounded by a medium having a lower refractive index. Typically, this medium is a relatively thin cladding, which is an intimately bound layer surrounding the core. The cladding may be a different material than the core, or it may be a similar material that has been doped in order to reduce its refractive index. The core may also be unclad whereby the ambient medium is of lower refractive index and acts in the capacity of the cladding. The cladding is typically surrounded by one or more coatings, buffers, and/or jackets that primarily serve protective roles.

Preferably, the light transporter is cylindrical in shape, of any desired length, and its diameter is as small as possible while retaining the ability to emit light. The light transporter can be operably connected to (in operable communication with) a light source. The light transporter can have a proximal end and a distal end (free end), such as a segment of optical fiber. The proximal end of the light transporter is connected to the light source, such that light energy is provided by the light source into the light transporter at its proximal end (also referred to as its input end) and travels to its distal end (also referred to as its output end), where it is emitted.

Preferably, at least a segment of the light transporter is flexible. More preferably, at least the distal end of the light transporter is flexible. Flexibility of the distal end allows versatility in the path of insertion within the eye. Optionally, the distal end of the light transporter can include a means for handling the light transporter, such as a handpiece, which can be made of rubber, plastic, or other material. Preferably, the handling means is rigid. The handling means can be any size or shape. Preferably, the handling means is sufficiently ergonomic so as to minimize tension within the hand of the operator of the apparatus. However, a minaturized form of the light transporter in which all segments are included in a single, thin, rigid cylindrical body is also possible.

The distal end of the light transporter can have a tip from which the light is emitted. The tip can have any of a variety of shapes, such as flat or planar, rounded, convex, concave, beveled, or tapered.

The intensity of the light emitted by the light emitting means of the invention may have various wavelengths and energy (intensity or power). The established limits of both as recommended or allowed by the Food and Drug Administration (FDA) are desired in order to guarantee safety. Preferably, the energy transmitted into the eye and the lens shall be as low as is necessary to visualize the target feature without damaging the tissues of the eye. Other optical devices and computer programs can be utilized to enhance the illumination achieved with the invention and may further decrease the amount of light emission energy necessary. In one embodiment, the light's energy can be adjusted such that is increased or decreased. In a specific embodiment, the light's energy can be increased to an extent that it is capable of, and appropriate for, surgical incision, and can be decreased to an extent that it does no damage to surrounding tissues yet provides improved visualization of the lens and anterior lens capsule.

Various materials and methods for optical fiber light manipulation can be utilized in conjunction with the light emitting means of the subject invention, such as those materials and methods described in relation to the fiber optic probes disclosed in U.S. Pat. No. 6,487,349 (Wach et al.), the contents of which is incorporated by reference herein in its entirety.

The apparatus of the subject invention can exist independently or can be a component of a more comprehensive apparatus used for ophthalmologic surgery and/or study, such as a surgical cutting device, an aspiration device, a surgical microscope, and/or a surgical camera.

In a specific embodiment, the subject invention involves a method for visualizing the anterior lens capsule and the lens of a human subject, wherein the method comprises introducing a means for emitting light into the eye, wherein the light emitting means is a light transporter or light transporting medium, such as an optical fiber, and wherein, preferably, the light emitted is laser light.

In accordance with another aspect of the invention, there is provided a method for protecting the macula and the retina of the eye from direct illumination, wherein the method comprises introducing a means for emitting light into the lens of the eye, wherein the light emitting means is a light transporter or light transporting medium, such as an optical fiber, and wherein the light emitting means is inserted into eye from a direction that is not coaxial with the retina.

As used herein, the phrase “visualization of the lens” and/or “visualization of the “anterior lens capsule” means to use a light emitting means, as described herein, in order to obtain an enhanced view of the lens and/or anterior lens capsule surface and features within the lens and/or anterior lens capsule.

The anatomy of the eye is well established and is comprehensively described, for example, in Duke-Elder S and Wybar K C. System of Ophthalmology, Volume II, The Anatomy of the Visual System; and Henry Kimpton, London 1961 and Bron A J, Tripathi R, Tripathi B. Wolff, Anatomy of the Eye and Orbit, Lippincott-Raven, 1998, the contents of which are incorporated herein by reference in their entirety. For the present purposes, the eye includes a pupil margin, defined by the iris, which regulates access of light to the lens. Pupil dilation caused by contraction of the iris dilator muscle defines a pupil margin and an area without the pupil overlying the lens.

The nature of the viscoelastic substances or their use during surgery is not critical to this invention or its use. There have been certain techniques described which relate to carrying out capsulorhexis processes which includes filling the anterior chamber with air. Such techniques are suboptimal as air contact causes corneal endothelial cell damage and may result in comeal edema. Generally, the pupil is dilated prior to the process of this invention, utilizing well-described agents for pupil dilation. Topical and peribulbar anaesthetic is conventionally used to anaesthetise the eye. One or more very small corneal-limbal incisions are made into the eye through which needles are introduced both for application of local anesthetics and, thereafter, viscoelastics. Through these openings then the terminal part of this invention is introduced into the eye. No additional surgical step is required. A needle or forceps for capsulorhexis can be readily inserted through a major incision at another location that is thereafter used for other surgical proceedings, such as the insertion and use of other instruments (such as phacoemulsification probes and an intraocular lens). All incisions are generally self-sealing and require no suture, or on some occasions only a single suture. Examples of ophthalmic techniques that may be utilized with visualization methods of the subject invention include those described in U.S. Pat. No. 6,367,480 (Coroneo) and U.S. Pat. No. 5,549,632 (Lai), the contents of which are incorporated herein by reference.

This invention in its various aspects is applicable to lenses, and particularly cataractous lenses, of humans and non-human animals, such as, dogs and cats.

### EXAMPLE 1

### Capsulorhexis (CCC) Conducted Utilizing the Invention

Following topical anesthesia with drops and insertion of an eyelid holder, an initial opening is created paralimbally at 10 o'clock with a sharp 15 degree knife. Intracameral anesthetics are applied via a syringe, inserted through this opening and allowed to excert effect for about 20 seconds. The syringe is removed and another syringe with a specific tip is introduced via the same opening into the anterior chamber. Viscoelastics are applied through this syringe which is withdrawn as soon as the viscous material has filled well the anterior chamber and replaced the her normally residing anterior chamber fluid nearly completely. Then a larger opening between approximately the 11 o'clock and 1 o'clock positions is performed with a sharp knife, approximately 2 to 3 mm in length, forming a self-sealing wound. The phacoilluminator tip is introduced via the paracentesis into the anterior chamber into closest proximity of the lens, the light is turned on. With this the formerly white lens or part of it becomes illuminated with red light (in the case that a Helium-Neon laser is used as the means for light emission). Next, the anterior capsule is perforated with a needle or a knife and a circular curvilinear capsulorhexis (CCC) is performed with forceps or a bent needle, introduced into the eye via the larger opening. The edge of the CCC is clearly visible as a sharp, dark line against the red illumination around. During the entire operation the tip of the phacoilluminator may follow the proceeding edge of the CCC until it is complete or it may be stable, depending on the light energy used and the opacity and optical properties of the cataractous lens. When the CCC is completed, the excised capsule is taken from the anterior chamber via the larger opening with forceps. Now follow the well established steps of surgery such as phacoemulsification, cortex aspiration and lensinsertion, as well documented and described in the specific text books and journals of ophthalmic surgery.

Throughout this specification and the claims which are stated above or will follow, unless the context requires otherwise, the word “comprise”, or variations such as “comprises” or “comprising”, will be understood to imply the inclusion of a stated integer or step or group of integers or steps but not the exclusion of any other integer or step or group of integers or steps.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

What is claimed is:

1. A method for visualizing the lens and/or anterior lens capsule of a subject, comprising introducing a means for emitting light into the lens of the subject's eye, wherein light emitted by the light emitting means illuminates the lens and/or anterior lens capsule of the eye by light scattering and reflection.

2. The method of claim 1, further comprising visualizing the illuminated lens and/or anterior lens capsule of the eye.

3. The method of claim 1, wherein the emitted light has an angle of dispersion between about 0 degrees and about 20 degrees, thereby achieving a higher local scattering of the emitted light in the lens.

4. The method of claim 1, wherein the light emitted by the light emitting mean is directed through the eye at an angle that is not coaxial to the optical axis of the eye.

5. The method of claim 1, wherein the light emitted by the light emitting means is laser light.

6. The method of claim 1, wherein said introducing comprises inserting the light emitting means into the anterior chamber of the eye.

7. The method of claim 1, wherein the light emitting means comprises a light transporter, wherein the light transporter comprises a proximal end and a distal end, and wherein the distal end is inserted into the anterior chamber of the eye.

8. The method of claim 7, wherein the distal end of the light transporter has a tip from which light is emitted, wherein the distal end of the light transporter inserted into the anterior chamber, and wherein the tip of the distal end of the transporter is within the range of about 0 millimeters to about 15 millimeters from the lens of the eye.

9. The method of claim 1, wherein the light emitting means comprises a light transporter, wherein the light transporter comprises a proximal end and a distal end, and wherein the distal end is inserted through the anterior chamber of the eye and into the lens of the eye.

10. The method of claim 9, wherein the light transporter is an optical fiber.

11. The method of claim 1, wherein the light emitting means comprises a light transporter, wherein the light transporter comprises a proximal end and a distal end, wherein the distal end is inserted through the anterior chamber of the eye and into the lens of the eye prior to, during, or after a surgical procedure on the eye.

12. A method for visualization of the lens and/or the anterior lens capsule of the eye during a capsulorhexis procedure, comprising introducing a viscoelastic substance into the anterior chamber of the eye, inserting a means for emitting light into the lens of the subject's eye, wherein light emitted by the light emitting means illuminates the lens and/or anterior lens capsule of the eye by light scattering and reflection, and creating a capsulorhexis within the eye.

13. The method of claim 12, wherein the emitted light is directed at the retina, and wherein the emitted light is reflected off the retina and illuminates the lens and/or anterior lens capsule of the eye.

14. The method of claim 12, wherein the emitted light has an angle of dispersion, between about 0 degrees and about 20 degrees, thereby achieving a higher local scattering of the emitted light in the lens.

15. The method of claim 12, further comprising replacing at least a portion of the lens with an intraocular lens.

16. The method of claim 12, wherein viscoelastic substances of differing viscosities are introduced into the eye, wherein a less viscous substance is introduced over the lens capsule, and wherein a more viscous substance is introduced at the pupil margin of the eye so as to enhance visibility of the edge of the capsulorhexis.

17. The method according to claim 12, wherein viscoelastic substances of differing viscosities are introduced into the eye, wherein a more viscous substance is introduced over the lens capsule and at the pupil margin of the eye so as to enhance visibility of the edge of the capsulorhexis.

18. An apparatus for the visualization of the lens and/or anterior lens capsule of the eye, comprising a light transporter and a light source, wherein said light transporter is operably connected to said light source, and wherein said light transporter emits light.

19. The apparatus of claim 18, wherein said light transporter has a proximal end connected to said light source, and wherein said light transporter has a distal end, wherein light is emitted from said distal end of said light transporter.
